(19) United States
(12) Patent Application Publication
(10) Pub. No.: US 2025/0255995 A1
(43) Pub. Date: Aug. 14, 2025

(54) ULTRAVIOLET BIOFLUID AND FLUID IRRADIATING DEVICE

(71) Applicants: James K. Burgess, III, Edmond, OK (US); Mark Chivers, Fairview, TX (US); Phil Chivers, Plano, TX (US); Josh Anderson, McKinney, TX (US); Sujit Ravindran, McKinney, TX (US); Mark Wood, Plano, TX (US); Scott A Cohen, Paradise Valley, AZ (US); Tom Sheffield, Hackett, AR (US)

(72) Inventors: James K. Burgess, III, Edmond, OK (US); Mark Chivers, Fairview, TX (US); Phil Chivers, Plano, TX (US); Josh Anderson, McKinney, TX (US); Sujit Ravindran, McKinney, TX (US); Mark Wood, Plano, TX (US); Scott A Cohen, Paradise Valley, AZ (US); Tom Sheffield, Hackett, AR (US)

(21) Appl. No.: 18/438,391

(22) Filed: Feb. 9, 2024

(51) Int. Cl. A61L 2/00; A61L 2/24

(52) U.S. Cl. CPC A61L 2/0047; A61L 2/24; A61L 2202/14; A61L 2202/16; A61L 2202/22

(57) ABSTRACT

This invention presents a portable, battery-operated ultraviolet biofluid and fluid irradiating device, designed for deactivating pathogens in biofluids and non-terrestrial ice slurry or soil using UV LEDs. Ideal for battlefield, emergency scenarios, and low gravity or no gravity scenarios such as space travel and non-terrestrial environments. It features a microcassette for biofluid handling and PCR analysis with or without necessity for adjuvants. The device is compact, lightweight, rugged, and suitable for rapid transfusions, revolutionizing field medical care with its innovative and versatile technology which may further be adapted for non-terrestrial ice/fluid processing.

### BACKGROUND

The current landscape of biofluid irradiation technologies encompasses a range of devices and methods primarily developed for use in controlled, clinical environments. These technologies are generally designed for sterilizing biofluids, such as blood products, to ensure their safety before medical procedures like transfusions. Common methods include the use of chemicals, radiation, or filtration systems to deactivate or remove pathogens. However, these existing technologies exhibit several limitations when considered for field deployment, especially in military, emergency and disaster scenarios.

Size and Portability: Many of the current biofluid irradiation devices are bulky and not designed for easy transport. Their size and weight make them impractical for use in field conditions where mobility and rapid deployment are crucial.

Dependency on External Power Sources: A significant limitation of current technologies is their reliance on stable electrical power sources. This dependency renders them ineffective in remote or battlefield or low gravity environments where electricity supply is often unreliable or unavailable.

Complexity in Operation: These devices often require trained personnel to operate, due to their complexity and the precision needed in handling biofluids. This requirement poses a challenge in emergency situations where skilled operators may not be available.

Use of Chemicals for PCR Analysis: Current methods for preparing biofluids for Polymerase Chain Reaction (PCR) analysis often involve the addition of chemicals, adjuvants or reagents. This necessity complicates the process, especially in time-sensitive situations and where supply chain constraints exist.

Inadequacy for Immediate Field Use: Most existing biofluid irradiation methods are not designed for immediate use in field conditions, such as battlefields or disaster zones. This gap in capability presents a significant challenge in providing timely and safe medical care in such scenarios.

Given these limitations, there exists a pressing need for a device that overcomes these challenges, particularly in the context of field medical care requiring use of portable equipment. The proposed invention aims to fill this gap by providing a solution that is:
Compact and Portable: The new device is designed to be small, lightweight, and easy to transport, making it ideal for use in various field conditions where quick deployment is essential.
Battery-Operated: By operating on battery power, (as well as AC power), the device overcomes the limitation of requiring external power sources, making it highly suitable for use in remote locations and in situations where electricity supply is uncertain.
Rugged and Reliable: Considering the harsh conditions of battlefields and disaster areas, the device is built to be rugged, lightweight and able to withstand challenging environments, ensuring reliability when it's needed most.
Simplified Operation for Field Use: The device's design focuses on ease of use, allowing personnel with minimal training to operate it effectively in emergency and challenging situations including that of the battlefield.
Direct Biofluid Handling for PCR Analysis: A key innovation of this device is its ability to handle biofluids for PCR analysis directly, without the need for additional adjuvant chemicals. This capability is crucial for rapid diagnostics and treatment in the field, especially for immediate transfusions and other critical medical procedures.
Immediate Application in Battlefield Conditions: Specifically tailored for emergency and battlefield use, the device addresses the unique demands of military medical care and civilian emergency medical care, offering a solution that can significantly improve the safety and effectiveness of emergency medical interventions in these challenging environments. This device can also be used in low gravity or no gravity environments such as on a space station, the moon, Mars and in-transit space travel.

In summary, the proposed invention addresses the critical needs unmet by current technologies, offering a practical, efficient, and innovative solution for biofluid irradiation in field conditions, revolutionizing the approach to medical care in these contexts.

### TECHNICAL FIELD

The present invention relates to the field of medical devices and technologies, with a particular focus on portable, field-deployable medical equipment. It introduces a novel device designed for the irradiation of biofluids using ultraviolet (UV) light, primarily intended for the inactivation of pathogens. The pathogens inactivated include including viruses, bacteria, fungi, and parasites. This invention falls under the broader category of medical devices used for diagnostic and therapeutic purposes in challenging environments including a battlefield environment.

The uniqueness of this invention lies in its specific adaptation for use in field conditions, such as those encountered in military operations, disaster zones, or remote areas where traditional medical infrastructure is unavailable or impractical also including low gravity environments such as on the Moon or Mars. The device is characterized by several key features that make it especially suitable for these environments:
Portability: The device is designed to be easily transportable, with considerations for size, weight, and ease of handling. This portability ensures that it can be quickly and efficiently moved to and used in various locations where medical aid is urgently needed, such as battlefields, remote areas or low gravity environments.
Battery Operation: Understanding the constraints of field conditions, where access to stable electrical power sources is often limited or non-existent, the device is equipped with a battery-operated power system and can also use an available AC power source. The battery-operated power system allows for its use in off-grid situations and ensures continuous operation even in the absence of traditional power sources.
Ultraviolet Biofluid Irradiation: The core function of the device is to irradiate biofluids—such as blood, plasma, and other bodily fluids—with ultraviolet light. UV irradiation is a well-established method for inactivating a wide range of pathogens, including viruses, bacteria, fungi, and parasites. The ability to perform such irradiation in the field is crucial for ensuring the safety of medical procedures, particularly transfusions and other biofluid handling processes including the ability to monitor UV radiation for quality control purposes. Further functions of the device is to enhance compliance with Food and Drug Administration (FDA) or other regulatory agencies compliance issues with sensors and computational requirements to regulate such UV radiation exposure for compliance purposes
Pathogen Deactivation: The device utilizes UV light in specific wavelengths known to be effective in deactivating pathogens, rendering them non-biologically harmful. This is of paramount importance in emergency medical situations, where the risk of infection and disease transmission is heightened, and conventional sterilization methods are not feasible. This technology is uniquely adapted to render as yet unidentified pathogens that may be found in non-terrestrial ice and or soil as non-biologically harmful to humans through the use of interchangeable microcassettes, each designed for a specific task in the ice processing chain. The primary modification involves the development of two distinct types of microcassettes: one for converting ice slurry into water (melting and initial processing) and another for irradiating and purifying the resulting water (final processing). This dual-device configuration allows for a modular and flexible approach to ice processing in space, with each unit powered by a DC transformer and compactly sized for efficiency and ease of integration into space habitats.
Suitability for Battlefield Use: The device is explicitly designed for use in battlefield conditions, which are often characterized by their urgency, harshness, and unpredictability. This requires the device to be not only portable and battery-operated but also rugged, reliable, and easy to use under stressful and time-sensitive conditions.
Compatibility with PCR Analysis: An innovative aspect of this device is its compatibility with Polymerase Chain Reaction (PCR) analysis, enabling the processing of biofluids for direct re-injection into patients without the need for addition of chemical adjuvants. This feature is particularly beneficial for rapid diagnostics and treatment in field conditions. The device is also suitable for industrial applications such as processing and or reprocessing of fluids without the need for adjuvants. This can be a free-standing device or incorporated as a module in a larger system. Such device can also be used to process and or reprocess fluids in applications in low-gravity or no gravity environments such as in non-terrestrial, space environments.

In summary, the invention represents a significant advancement in the field of medical devices, particularly those designed for emergency and field medical care. It combines portability, ease of use, and the effective deactivation of pathogens through UV irradiation, addressing critical needs in situations like battlefield medical care, disaster response, and remote medical interventions.

### SUMMARY

The following summary is provided to introduce a selection of concepts in a simplified form that are further described below in the detailed description. This summary is not intended to identify key features or essential features of the claimed subject matter, nor is it intended to be used to limit the scope of the claimed subject matter.

In various implementations, an ultraviolet biofluid irradiating device is provided. The device comprises a plurality of Ultraviolet Light Emitting Diodes (“UV LEDs”) for ultraviolet biofluid irradiating (“UBI”), emitting ultraviolet light in the range of 100 nm to 380 nm. The device comprises a control circuitry for regulating the ultraviolet light emission by said UV LEDs. A housing is constructed with material capable of ultraviolet light transmittance, housing said UV LEDs and said control circuitry. The device comprises a UV transmissible microfluidics device including a microcassette capable of biofluid handling techniques, enabling the device to process said biofluid for analysis for direct injection into a human. The microcassette, which is a biofluid conveying conduit, is configured such that the biofluid flows through a tube which allows aid biofluid can be irradiated with ultraviolet light emitted from said UV LEDs as it is flowing through the microcassette. The device is powered by a power source that allows it to be used both in and out of clinical settings. The device is configured such that pathogens in the biofluid are rendered non-biologically harmful as they are irradiated with UV light while flowing through the microcassette in the device, then direct injecting said biofluids rendered non-biologically harmful into the human.

These and other features and advantages will be apparent from a reading of the following detailed description and a review of the appended drawings. It is to be understood that the foregoing summary, the following detailed description and the appended drawings are explanatory only and are not restrictive of various aspects as claimed.

### BRIEF DESCRIPTION OF THE DRAWINGS

FIG. 1 is a block diagram of an ultraviolet biofluid irradiation device in accordance with the subject disclosure.

FIG. 2 is a block diagram of components of the ultraviolet biofluid irradiation device in accordance with the subject disclosure.

FIG. 3 is a block diagram of another embodiment of the ultraviolet biofluid irradiation device in accordance with the subject disclosure.

FIG. 4 is a block diagram of another embodiment of the ultraviolet biofluid irradiation device in accordance with the subject disclosure.

FIG. 5 is a block diagram of another embodiment of the ultraviolet biofluid irradiation device in accordance with the subject disclosure.

FIG. 6 is a block diagram of a process utilizing the ultraviolet biofluid irradiation device in accordance with the subject disclosure.

### DETAILED DESCRIPTION

The detailed description provided below in connection with the appended drawings is intended as a description of examples and is not intended to represent the only forms in which the present examples can be constructed or utilized. The description sets forth functions of the examples and sequences of steps for constructing and operating the examples. However, the same or equivalent functions and sequences can be accomplished by different examples.

References to “one embodiment,” “an embodiment,” “an example embodiment,” “one implementation,” “an implementation,” “one example,” “an example” and the like, indicate that the described embodiment, implementation or example can include a particular feature, structure or characteristic, but every embodiment, implementation or example can not necessarily include the particular feature, structure or characteristic. Moreover, such phrases are not necessarily referring to the same embodiment, implementation or example. Further, when a particular feature, structure or characteristic is described in connection with an embodiment, implementation or example, it is to be appreciated that such feature, structure or characteristic can be implemented in connection with other embodiments, implementations or examples whether or not explicitly described.

References to a “module”, “a software module”, and the like, indicate a software component or part of a program, an application, and/or an app that contains one or more routines. One or more independent modules can comprise a program, an application, and/or an app.

References to an “app”, an “application”, and a “software application” shall refer to a computer program or group of programs designed for end users. The terms shall encompass standalone applications, thin client applications, thick client applications, mobile-based applications, web-based applications, such as a browser, and other similar applications.

Numerous specific details are set forth in order to provide a thorough understanding of one or more embodiments of the described subject matter. It is to be appreciated, however, that such embodiments can be practiced without these specific details.

The subject disclosure is directed to an ultraviolet biofluid irradiation device comprising UV LEDs, control circuitry, housing, and microfluidics device and microcassette. Each component is further described herein:

### UV LEDs:

Specification: The UV LEDs are the primary component for irradiating the biofluids. These LEDs are designed to emit ultraviolet light in the specific range of 100 nm to 380 nm, with an optimal range of 250 nm to 320 nm for effective pathogen deactivation.

Function: The selected wavelength range is crucial as it targets the nucleic acids of pathogenic microbes, causing damage to their DNA or RNA, rendering them inactive without damaging the biofluid itself.

Design Considerations: The LEDs are engineered to be energy-efficient and durable, suitable for prolonged use in field conditions. Their compact size contributes to the overall portability of the device.

### Control Circuitry:

Overview: The control circuitry is the central system regulating the operation of the device. It includes several key functions. The circuitry may include wireless and data ports, data storage memory components on the device, a microcontroller, a clock, and input/output functions.
Pulse Width Modulation (PWM): This feature allows for precise control over the intensity of UV light, enabling the device to adjust the irradiation based on specific requirements.
Temperature Control: Essential for maintaining optimal operating conditions for both the UV LEDs and biofluids, preventing overheating that could damage components or alter biofluid properties.
Intensity Regulation: Regulates the strength of UV light, ensuring sufficient irradiation to deactivate pathogens while maintaining the integrity of the biofluids.
Pump Control: Manages the flow of biofluids through the microcassette, ensuring even exposure to UV light.
Exposure Duration: Controls the time biofluids are exposed to UV light, critical for ensuring effective pathogen deactivation.

### Housing:

Material Selection: The housing of the device is made from a UV-resistant polymer, such as polypropylene, known for its durability, resistance to chemical and UV damage, and suitability for medical applications.
Properties: The chosen material is lightweight yet sturdy, adding to the portability and ruggedness of the device. Its UV transmittance capability ensures that the UV light effectively reaches the biofluids.

### Microfluidics Device and Microcassette:

Microfluidics Device: This component is integral to handling and processing biofluids. It's designed to be compact and efficient, capable of managing small volumes of fluid with high precision.
Microcassette: A key innovation, the microcassette facilitates the flow of biofluids, exposing the biofluids evenly to UV light. It's designed for easy insertion and removal, and can be disposable for hygiene, avoidance of contamination from biofluids and convenience.
Biofluid Handling and PCR Analysis: The microcassette enables advanced biofluid handling techniques necessary for PCR analysis. This functionality allows the device to process biofluids for re-injection without needing additional chemicals, a significant advancement in the field.

### Interactions and Integration:

The components of the ultraviolet biofluid irradiating device are intricately designed to work in unison, ensuring efficient and effective operation:

The UV LEDs provide the necessary irradiation, the intensity and duration for the deactivation of pathogens, which are precisely controlled by the Control Circuitry.

The Housing Material not only protects the internal components but also facilitates the effective transmission of UV light.

The Microfluidics Device, particularly the Microcassette, plays a critical role in handling the biofluids, ensuring they are uniformly exposed to UV light and that the biofluids flow from one area to another.

The integrated design ensures that all components are optimized for portability, efficiency, and ease of use, especially in field conditions such as on battlefields, emergency situations and low-gravity situations.

This detailed description of the invention's components and their operation highlights the innovative integration of technology to achieve a compact, efficient, and field-appropriate device for biofluid irradiation and PCR analysis.

Various features of the subject disclosure are now described in more detail with reference to the drawings, wherein like numerals generally refer to like or corresponding elements throughout. The drawings and detailed description are not intended to limit the claimed subject matter to the particular form described. Rather, the intention is to cover all modifications, equivalents and alternatives falling within the spirit and scope of the claimed subject matter.

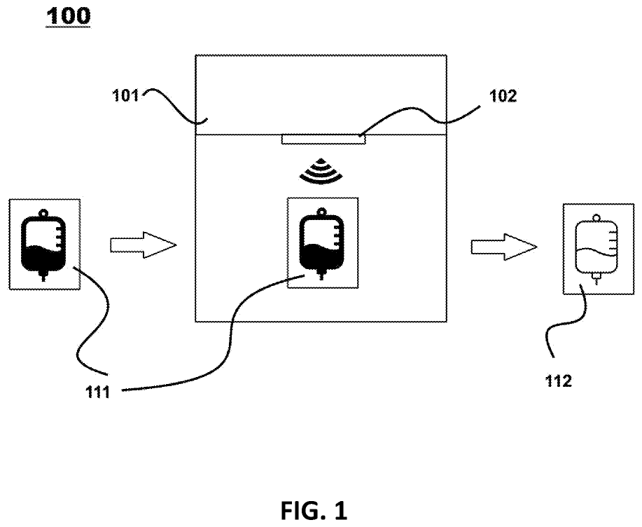

Now referring to the drawings and particularly to FIG. 1, various features of the subject disclosure are now described in more detail with respect to an ultraviolet biofluid irradiation device, generally designated 100. The ultraviolet biofluid irradiation device 100 comprises a housing 101 with a plurality of light emitting diodes (LEDs) 102. The housing is constructed from an ultraviolet (UV) resistant polymer, particularly polypropylene. The material is known for its durability, resistance to chemicals and UV damage, and suitability for medical applications. The housing 101 is lightweight and sturdy, providing portability and ruggedness to the UV biofluid irradiation device 100.

The plurality of LEDs 102 are the primary components for irradiating biofluids. The LEDs 102 are designed to emit UV light in specific range of 100 nm to 380 nm in the exemplary embodiment. An optimal range of 260 nm to 280 nm is found to be effective for pathogen deactivation. The UV biofluid irradiation device 100 is designed to regulate the LEDs 102 in regards to range of wavelength in order to target specific pathogens during application. Depending on the specific requirements, each selected wavelength range is crucial in targeting the nucleic acids of pathogenic microbes. This causes damage to the relevant DNA and RNA of the microbes, rendering them inactive without damaging the biofluid itself. The LEDs 102 are engineered to be energy efficient and durable, which makes them suitable for prolonged use in field conditions. The LEDs 102 are designed to be compact in order to facilitate portability of the UV biofluid irradiation device 100.

The UV biofluid irradiation device 100 comprises a plurality of microfluidics devices 111-112. The microfluidics devices 111-112 are in the form of microcassettes in the exemplary embodiment. The microcassettes are designed to be compact, efficient, and capable of managing small volumes of fluid with high precision. The microcassettes facilitate the flow of biofluids, such that the biofluids are exposed to the UV light emitted by the LEDs 102 evenly. The microfluidics devices 111-112 are designed for easy insertion and removal. These devices are also made to be easily disposable, thus drastically reducing contamination tasks.

In at least one exemplary embodiment, the microfluidics devices 111-112 enable advanced biofluid handling techniques necessary for polymerase chain reaction (PCR) analysis. This functionally allows the device 100 to process biofluids for re-injection without needing additional chemicals, thereby enabling significant applicability in the field.

In application, a microfluidics device 111, in the form of a microcassette, is configured with a certain volume of biofluids. The biofluids can be used for analysis for injection use with patients in need. In practice, the biofluids should be processed such that harmful pathogens are removed. Such pathogens can be from a group consisting of protozoans, bacteria, fungi and viruses. The protozoans can comprise that of malaria, African trypanosomiasis, Chagas disease, toxoplasmosis, cryptosporidiosis, amoebic dysentery and giardia. The bacteria can comprise that of salmonella, lyme disease, streptococcus, staphylococcus, methicillin-resistant Staphylococcus aureus (MRSA), Clostridium tetani, E. coli, campylobacter and pneumonia pneumococcus. The fungi can comprise that of Candida albicans, fungal meningitis, Cryptococcosis, Aspergillosism, pneumocystis pneumonia and histoplasmosis. Finally, the viruses can comprise of West Nile virus, Zika virus, Avian influenza A virus, Dengue virus, human SARS coronavirus (SARS-CoV), human SARS coronavirus 2 (SARS-CoV-2), MERS coronavirus (MERS-CoV), chikungunya virus and rabies virus and potentially currently unknown pathogens—protozoa, bacteria, fungi and viruses which may be present in ice and soil on non-terrestrial environments.

The microfluidics device 111 containing potential harmful pathogens is inserted into the housing 101 of the UV biofluid device 100. The device 100 is configured to regulate the UV exposure on the microcassette 111 in order to optimally eliminate the harmful pathogens from the biofluid contained within. The regulation of the UV exposure to the biofluids can be achieved in a number of ways. In the exemplary embodiments, the regulation can be enabled through the setting on the housing 101 and the LEDs 102, or alternatively through components on the microcassette 111.

In one exemplary embodiment, a high transmissible plastic film is used on the microcassette 111, wherein the transmissibility of the UV radiation can be enabled through a custom film. Therefore, different microcassettes 111 can be designated for different types of pathogen removals. Alternatively, a transmissivity hindrance component can be placed over the cartridge, thus allowing the microcassettes 111 the ability to have variable LED exposure using the hindrance device. The hindrance device can be in a form akin to lenses on sunglasses, such that a modified wavelength can be made to penetrate the microcassettes 111. As such, a combination of the LED hindrance device and transmissible plastic film of the microcassette 111 provide a customizable UV exposure set up. Utilizing the same concept, a plurality of hindrance components or films can be placed to fit over the LEDs 102. Thus, minimum hardware adjustment is required on the actual output of the LEDs 102 in order to produce a wide variety of wavelengths ranges by utilizing the variable films.

In other embodiments, the UV radiation intensity and range can adjusted with pulse width modulation (PWM) on the LEDs 102. Under certain operating parameters, the LEDs 102 are configured to run at 100% output, wherein variation of UV exposure on the microcassette 111 are achieved through a plurality of films, screens, or other hinderance components. In various embodiments, the UV biofluid irradiation device 100 can be configured to vary the power to the LEDs 102. This can be achieved by turning individual LEDs on and off, in one instance. This ability can be further configured to do a combination of lights (on and off, power up and down, and pulse with modulation) to enable different behaviors. Thus, the UV radiation output can be controlled from the source. When used in conjunction with the films or with modified cartridges 111, a greater variety of UV penetration can be enabled and changed on the fly. This provides significant customizability and flexibility for application using the device 100 outside of clinic settings.

In at least one embodiment, all of the changes described herein in the operation can be dynamically tied into a database. The database can be reached wired or wirelessly or even at the quantum level. Based on information within the database, the input could be a variety of different types of known sensors today, future sensors, or some sort of manual sensor or pin, such as a coleco quiz whiz, which is mentioned as a particular sensor.

Utilizing any number of UV radiation regulation methods, the UV biofluid irradiation device 100 can irradiate the biofluids within the microfluidics device 111 using the LEDs 102. After a particular period of irradiation exposure, the microfluidics device 111 can be extracted. The irradiated microfluidics device 111 becomes processed microfluidics device 112, which now contains biofluids that are no longer containing any harmful pathogens. The biofluid within the processed microfluidics device 112 can now be used for analytics. Alternatively, the biofluid within the processed microfluidics device 112 can be injected into patients with or without additional adjuvant chemicals.

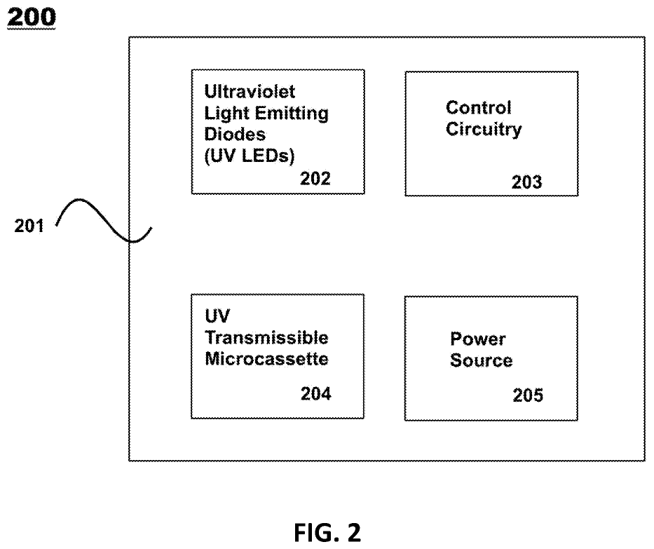

Referring to FIG. 2, a block diagram of the ultraviolet biofluid irradiation device is shown and generally designated 200. The UV biofluid irradiation device comprises a housing 201, a plurality of UV LEDs 202, a control circuitry 203, a UV transmissible microcassette 204, and a power source 205. The plurality of UV LEDs 202 can be the LEDs 102 of FIG. 1. The UV transmissible microcassette 204 can be the microfluidic devices 111-112 of FIG. 1. The control circuitry 203 is the central system regulating the operation of the device 200. The control circuitry 203 is configured to enable pulse width modulation (PWM), which allows for precise control over intensity of UV light emitted from the UV LEDs 202. The device 200 utilizes the control circuitry 203 to adjust the irradiation based on specific requirements of each application.

The control circuitry 203 is configured to provide temperature control within the components of the housing 201. The temperature control is essential in maintaining optimal operating conditions for both the UV LEDs 202 and the biofluids within the UV transmissible microcassettes 204. The device 200 utilizes temperature control to prevent overheating that would otherwise damage components within the housing 201 or alter properties of fluid within the microcassettes 204.

In at least one exemplary embodiments, the control circuitry 203 is configured to enable intensity regulation. This allows the control circuitry 203 to regulate strength of UV light emitted from the UV LEDs 202, ensuring sufficient irradiation to deactivate pathogens while maintaining the integrity of the biofluids during the process.

The control circuitry 203 is configured to control exposure durations, such that the effective pathogen deactivation can be achieved through sufficient exposure to the UV light emitted from the UV LEDs 202.

The power source 205 is designed and configured to enable the device 200 to be used in various field conditions. To overcome the constraints of filed conditions, where access to stable electrical power sources may be scarce, the device 200 is equipped with a battery-operated power system 205. The power source 205 can also be configured to utilized any available alternating current (AC) power source. The flexibility of the power source 2005 allows the device 200 to be used in off-grid situations and enables continuous operation in the absence of traditional power sources.

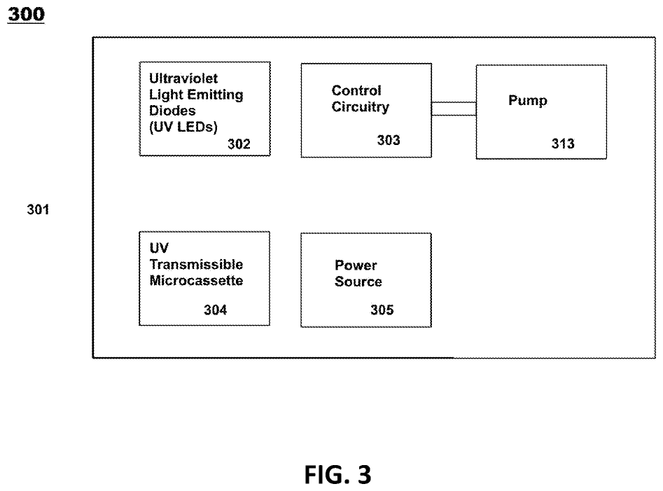

Referring to FIG. 3, a block diagram of another embodiment of the ultraviolet biofluid irradiation device is shown and generally designated 300. The UV biofluid irradiation device 300 comprises a housing 301, a plurality of UV LEDs 302, a control circuitry 303, a pump 313 connected to the control circuitry, a UV transmissible microcassette 304, and a power source 305. In this exemplary embodiment, the pump 313 is configured to manage flow of biofluids through the microcassette 304 to ensure even exposure to UV light. The pump 313 can coordinate with the control circuitry 303 to ensure that exposure duration is appropriate for the type of pathogen that the device 300 is working to remove. In various embodiments, the pump 313 is configured to operate in low-gravity situation, such that the device 300 can operate as intended in space settings. It is envisioned that the device 300 can be used on the surface of the moon or planet Mars.

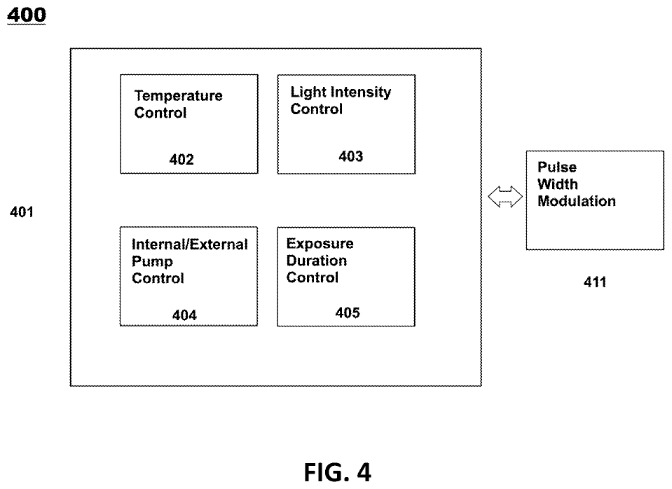

Referring to FIG. 4, a block diagram of another embodiment of the ultraviolet biofluid irradiation device is shown and generally designated 400. The UV biofluid irradiation device 400 comprises a housing 401, a plurality of UV LEDs 402, a control circuitry 403, a UV transmissible microcassette 404, and a power source 405. Further, the device 400 comprises a component for pulse width modulation 411. The control circuitry 403 is controlled by the pulse width modulation in this exemplary embodiment, wherein output of the UV LEDs 402 is further regulated by the pulse width modulation.

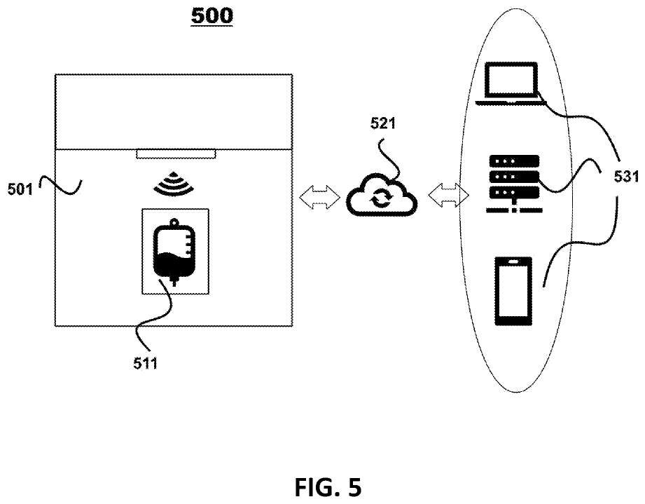

Referring to FIG. 5, another embodiment of the ultraviolet biofluid irradiation device is shown and generally designated 500. The device 501 is configured to irradiate and process the biofluid contained within the microcassette 511 in accordance to the preceding specification. In addition, the device 501 is configured to interface with a plurality of user devices 531. The user devices 531 can be one of mobile phone, personal computer, or computing servers. The user devices 531 interact with the UV biofluid irradiation device 501 through a wireless network 521 in the exemplary embodiment. In other embodiments, the user devices 531 are connected to the device 501 through wired communication.

As the device 501 is configured to regulate UV exposure duration, light intensity, and UV penetration, the instructions to these operations can be dynamically tied into a database that is accessible by the user devices 531. In various embodiments, the database accessible by the user devices 531 record the type of pathogens that the biofluid within the microcassette 511 may contain and issues instructions to the control circuitry on board the device 501. In various embodiments, a user can regulate and control the output of the UV LEDs through one of the user devise 531.

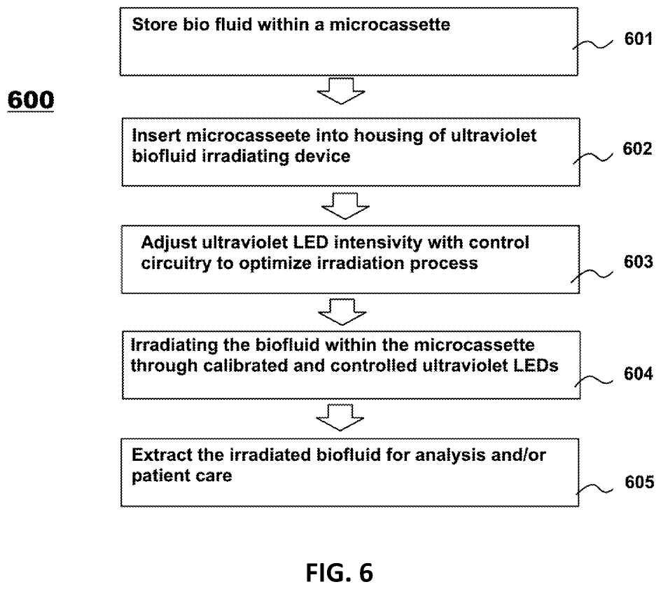

Referring to FIG. 6, a process utilizing the UV biofluid irradiation device is shown and generally designated as 600. At 601, biofluid is stored within a microcassette. The biofluid can be input into the microcassette at a clinical setting, a research setting, or a hospital setting. The microcassette provide the means for the biofluid to be safely stored and processed without compromising its quality or integrity.

At 602, the microcassette is inserted into a housing of the UV biofluid irradiation device. The receptacle of the microcassette can be oriented directly about a UV LED output, such that the biofluid can undergo irradiation processing through the microcassette.

At 603, the UV LED intensity is adjusted with the control circuitry within the UV biofluid irradiation device. The adjustment can be done in conjunction with filter, film, or radiation hinderance components to regulate the intensity, wavelength, and UV penetration through the microcassette.

At 604, the biofluid within the microcassette is irradiated through the UV LED output of the device. The duration, intensity, and UV penetration of the process is monitored and regulated with the control circuitry of the device. In various embodiments, the UV biofluid irradiation device is further controlled by a user through a user device, such that remote management of the device is feasible.

At 605, the irradiation is complete and the microcassette containing biofluid is ready to be removed. The biofluid within the microcassette should be free of harmful pathogens after undergoing irradiation processes. The microcassette is configured to interact directly with PCR devices for additional analysis. In various embodiments, the microcassette provides storage for the now processed biofluid, which can be injected for patient use in and out of clinical settings.

The detailed description provided above in connection with the appended drawings is intended as a description of examples and is not intended to represent the only forms in which the present examples can be constructed or utilized.

It is to be understood that the configurations and/or approaches described herein are exemplary in nature, and that the described embodiments, implementations and/or examples are not to be considered in a limiting sense, because numerous variations are possible.

The specific processes or methods described herein can represent one or more of any number of processing strategies. As such, various operations illustrated and/or described can be performed in the sequence illustrated and/or described, in other sequences, in parallel, or omitted. Likewise, the order of the above-described processes can be changed.

Although the subject matter has been described in language specific to structural features and/or methodological acts, it is to be understood that the subject matter defined in the appended claims is not necessarily limited to the specific features or acts described above. Rather, the specific features and acts described above are presented as example forms of implementing the claims.

The subject matter of the biofluid irradiating device is also readily adaptable to an ice slurry/fluid irradiating device which renders pathogens as non-harmful to humans in non-terrestrial ice slurry and soil in a non-terrestrial environment.

What is claimed is:

1. An ultraviolet biofluid irradiating device comprising:
a) Ultraviolet Light Emitting Diodes (“UV LEDs”) for ultraviolet biofluid irradiating (“UBI”), emitting ultraviolet light in the range of 100 nm to 380 nm;
b) control circuitry for regulating the ultraviolet light emission by said UV LEDs;
c) a housing material capable of ultraviolet light transmittance, housing said UV LEDs and said control circuitry;
d) a UV transmissible microfluidics device including a microcassette capable of biofluid handling techniques, enabling the device to process said biofluid for analysis for direct injection into a human;
f) said microcassette which is a biofluid conveying conduit in which said biofluid flows through a tube such that said biofluid can be irradiated with ultraviolet light emitted from said UV LEDs as it is flowing through the microcassette;
g) a power source;
such that pathogens in the biofluid are rendered non-biologically harmful as they are irradiated with UV light while flowing through the microcassette in the device, then direct injecting said biofluids rendered non-biologically harmful into the human.

2. The ultraviolet biofluid irradiating device of claim 1, wherein said pathogens are selected from the group consisting of protozoans, bacteria, fungi, viruses and currently unknown pathogens which may be present in ice and soil on non-terrestrial locations.

3. The ultraviolet biofluid irradiating device of claim 2, wherein said protozoans are selected from the group consisting of malaria, African trypanosomiasis, Chagas disease, toxoplasmosis, cryptosporidiosis, amoebic dysentery and giardia.

4. The ultraviolet biofluid irradiating device of claim 2, wherein said bacteria are selected from the group consisting of salmonella, Lyme disease, streptococcus, staphylococcus, methicillin-resistant Staphylococcus aureus (MRSA), Clostridium tetani, E. coli, campylobacter and pneumonia pneumococcus.

5. The ultraviolet biofluid irradiating device of claim 2, wherein said fungi are selected from the group consisting of Candida albicans, fungal meningitis, Cryptococcosis, Aspergillosism, pneumocystis pneumonia and histoplasmosis.

6. The ultraviolet biofluid irradiating device of claim 2, wherein said viruses are selected from the group consisting of West Nile virus, Zika virus, Avian influenza A virus, Dengue virus, human SARS coronavirus (SARS-CoV), human SARS coronavirus 2 (SARS-CoV-2), MERS coronavirus (MERS-CoV), chikungunya virus and rabies virus.

7. The ultraviolet biofluid irradiating device of claim 1, wherein said analysis of the biofluid is Polymerase Chain Reaction (“PCR”) analysis.

8. The ultraviolet biofluid irradiating device of claim 1, wherein said direct injection into said human is selected from the group consisting of direct injection without adjuvant chemicals and direct injection with adjuvant chemicals.

9. The ultraviolet biofluid irradiating device of claim 1, wherein said direct injecting said biofluids into said human can be done in non-medical room settings for soldiers in the field for wound care.

10. The ultraviolet biofluid irradiating device of claim 1, wherein the type of said flowing through the microcassette is selected from the group consisting of flowing caused by gravity and flowing caused by a pump to regulate the flowing through said device.

11. The ultraviolet biofluid irradiating device of claim 10, wherein said flowing caused by a pump to regulate the flowing through said device can be done in a low-gravity situation such as that on the surface of the moon or the planet Mars.

12. The ultraviolet biofluid irradiating device of claim 1, wherein said housing is selected from the group consisting of thermoform/injection-molded material for handling microfluidics and molded polymer material for handling microfluidics.

13. The ultraviolet biofluid irradiating device of claim 1, wherein said control circuitry is controlled by pulse width modulation (“PWM”).

14. The ultraviolet biofluid irradiating device of claim 1, wherein said control circuitry regulates temperature control.

15. The ultraviolet biofluid irradiating device of claim 1, wherein said control circuitry regulates the intensity of the ultraviolet light emission.

16. The ultraviolet biofluid irradiating device of claim 1, wherein said control circuitry regulates control of said pump (internal or external).

17. The ultraviolet biofluid irradiating device of claim 1, wherein said control circuitry selected from the group consisting of wireless and data ports, data storage memory components on the device, a microcontroller, a clock, and input/output functions which regulates the duration of exposure of the biofluid to UV radiation from the UV LEDs.

18. The ultraviolet biofluid irradiating device of claim 1, wherein said UV LEDs emit ultraviolet light in the range of 250 nm to 320 nm.

19. The ultraviolet biofluid irradiating device of claim 1, wherein said control circuitry further comprises a feedback mechanism for adjusting the emission intensity of said UV LEDs based on detected biofluid characteristics.

20. The ultraviolet biofluid irradiating device of claim 1, wherein said device is comprised of ultraviolet transmittance capable thermoform/injection molded UV-resistant polymer.

21. The ultraviolet biofluid irradiating device of claim 16, wherein said UV-resistant polymer is polypropylene.

22. The ultraviolet biofluid irradiating device of claim 1, wherein said UV transmittable Microfluidics device is capable biofluid handling techniques that comprise a disposable biofluid sampling cartridge capable of containing a biofluid sample and facilitating PCR analysis without the need for additional reagents or adjuvants.

23. The ultraviolet biofluid irradiating device of claim 1, wherein said device comprises a portable device.

24. The ultraviolet biofluid irradiating device of claim 1, wherein said device comprises a battery-powered device.

25. The ultraviolet biofluid irradiating device of claim 1, wherein said device comprises a compact device.

26. The ultraviolet biofluid irradiating device of claim 1, wherein said device comprises a lightweight device.

27. The ultraviolet biofluid irradiating device of claim 1, wherein said device comprises a sturdy device for use on a battlefield.

28. The ultraviolet biofluid irradiating device of claim 1, wherein the biofluid is selected from the group consisting of whole blood, plasma and platelets, plasma, platelets, red blood cells and white blood cells.

29. The ultraviolet biofluid irradiating device of claim 11, wherein said pump is regulated on a timing cycle for effectiveness in rendering pathogens non-biologically harmful.

30. The ultraviolet biofluid irradiating device of claim 1, wherein said control circuitry in the device affects coagulation factors in the biofluid such that Partial Thromboplastin Time (PTT) is effective for said direct injection into said human.

31. The ultraviolet biofluid irradiating device of claim 1, wherein said AC power source has a DC transformer.

32. The ultraviolet biofluid irradiating device of claim 1, wherein said pathogens are rendered non-biologically harmful by the irradiation as the phosphodiester bond between the pathogens' bonding group consisting of Uracil and Cytosine (U-C) and Uracil and Guanine (U-G) is broken.

33. The ultraviolet biofluid irradiating device of claim 1, wherein said power source is selected from the group consisting of a battery power source and AC power source.

34. The ultraviolet biofluid irradiating device of claim 1, wherein the control circuitry modifies individual UV LEDs to manage radiation intensity.

35. The ultraviolet biofluid irradiating device of claim 34, wherein the control circuitry is managed by a database accessible through a wired or wireless network.

36. An ultraviolet ice slurry/fluid irradiating device for use in non-terrestrial environments, comprising:
a) Ultraviolet Light Emitting Diodes (“UV LEDs”) for ultraviolet fluid irradiating (“UBI”), emitting ultraviolet light in the range of 100 nm to 380 nm;
b) control circuitry for regulating the ultraviolet light emission by said UV LEDs;
c) a housing material capable of ultraviolet light transmittance, housing said UV LEDs and said control circuitry;
d) a UV transmissible microfluidics device including interchangeable microcassettes capable of ice and or fluid handling techniques, enabling the device to process said ice and or fluid for analysis to determine presence of pathogens, wherein one microcassette is for converting ice slurry into water and wherein a second microcassette is for irradiating the liquid water with said UV LEDs,
f) said microcassettes which are fluid conveying conduits in which said fluid water flows through tubes such that said fluid can be irradiated with ultraviolet light emitted from said UV LEDs as it is flowing through the microcassettes;
g) a DC power source;
such that any pathogens in the ice slurry and or water are rendered non-biologically harmful as they are irradiated with UV light while flowing through the microcassettes in the device.

37. The ultraviolet ice slurry/fluid irradiating device of claim 36, wherein said ice/fluid irradiating device is compactly sized for efficiency and ease of integration in non-terrestrial environments.

38. The ultraviolet ice slurry/fluid irradiating device of claim 36, wherein said housing is selected from the group consisting of thermoform/injection-molded material for handling microfluidics and molded polymer material for handling microfluidics.

39. The ultraviolet ice slurry/fluid irradiating device of claim 36, wherein said control circuitry is controlled by pulse width modulation (“PWM”).

40. The ultraviolet ice slurry/fluid irradiating device of claim 36, wherein said control circuitry regulates temperature control.

41. The ultraviolet ice slurry/fluid irradiating device of claim 36, wherein said control circuitry regulates the intensity of the ultraviolet light emission.

42. The ultraviolet ice slurry/fluid irradiating device of claim 36, wherein said control circuitry regulates control of said pump (internal or external).

43. The ultraviolet ice slurry/fluid irradiating device of claim 36, wherein said control circuitry selected from the group consisting of wireless and data ports, data storage memory components on the device, a microcontroller, a clock, and input/output functions which regulates the duration of exposure of the fluid to UV radiation from the UV LEDs.

44. The ultraviolet ice slurry/fluid irradiating device of claim 36, wherein said UV LEDs emit ultraviolet light in the range of 250 nm to 320 nm.

45. The ultraviolet ice slurry/fluid irradiating device of claim 36, wherein said control circuitry further comprises a feedback mechanism for adjusting the emission intensity of said UV LEDs based on detected fluid characteristics.

46. The ultraviolet ice slurry/fluid irradiating device of claim 36, wherein said device is comprised of ultraviolet transmittance capable thermoform/injection molded UV-resistant polymer.

47. The ultraviolet ice slurry/fluid irradiating device of claim 46, wherein said UV-resistant polymer is polypropylene.

48. The ultraviolet ice slurry/fluid irradiating device of claim 36, wherein said device comprises a portable device.

49. The ultraviolet ice slurry/fluid irradiating device of claim 36, wherein said device comprises a battery-powered device.

50. The ultraviolet ice slurry/fluid irradiating device of claim 36, wherein said device comprises a compact device.

51. The ultraviolet ice slurry/fluid irradiating device of claim 36, wherein said device comprises a lightweight device.

52. The ultraviolet ice slurry/fluid irradiating device of claim 36, wherein said device comprises a sturdy device for use in a non-terrestrial environment.

53. The ultraviolet ice slurry/fluid irradiating device of claim 36, wherein the type of said flowing through the microcassette is selected from the group consisting of flowing caused by gravity and flowing caused by a pump to regulate the flowing through said device.

54. The ultraviolet biofluid irradiating device of claim 53, wherein said flowing caused by a pump to regulate the flowing through said device can be done in a low-gravity or no-gravity situation such as that on a non-terrestrial environment.

55. The ultraviolet ice slurry/fluid irradiating device of claim 36, wherein said pump is regulated on a timing cycle for effectiveness in rendering pathogens non-biologically harmful.

56. The ultraviolet ice slurry/fluid irradiating device of claim 36, wherein said power source is selected from the group consisting of a battery power source and AC power source.

57. The ultraviolet ice slurry/fluid irradiating device of claim 36, further comprising a plurality of UV filters, wherein the UV filters are compatible with the microcassette and the UV LEDs.

58. The ultraviolet ice slurry/fluid irradiating device of claim 36, wherein the control circuitry modifies individual UV LEDs to manage radiation intensity.

59. The ultraviolet ice slurry/fluid irradiating device of claim 58, wherein the control circuitry is managed by a database accessible through a wired or wireless network.

60. The ultraviolet ice slurry/fluid irradiating device of claim 36, wherein said pathogens are rendered non-biologically harmful by the irradiation as the phosphodiester bond between the pathogens' bonding group consisting of Uracil and Cytosine (U-C) and Uracil and Guanine (U-G) is broken.

61. The ultraviolet biofluid irradiating device of claim 1, further comprising a plurality of UV filters, wherein the UV filters are compatible with the microcassette and the UV LEDs.
